(19) United States
(12) Patent Application Publication
(10) Pub. No.: US 2022/0313423 A1
(43) Pub. Date: Oct. 6, 2022

(54) Ear Stent And Methods Of Using Same

(71) Applicant: United States Government as Represented by the Department of Veterans Affairs, Washington, DC (US)

(72) Inventors: Kent Flanagan, Charleston, SC (US); Bethany Baldwin, Charleston, SC (US); Nicole Beitenman, Charleston, SC (US)

(21) Appl. No.: 17/711,782

(22) Filed: Apr. 1, 2022

Related U.S. Application Data
(60) Provisional application No. 63/227,599, filed on Jul. 30, 2021.
(60) Provisional application No. 63/170,132, filed on Apr. 2, 2021.

(51) Int. Cl. A61F 2/18

(52) U.S. Cl. CPC A61F 2/18; A61F 2002/183; A61F 2250/0037; A61F 2230/0073

(57) ABSTRACT

An ear stent can be inserted into a collapsing ear canal to improve conductive hearing. The ear stent can be configured for insertion into an ear canal of a user. The ear stent can have a longitudinal axis. The ear stent can comprise an insertional portion that extends along the longitudinal axis between an insertional end and an outer end. The insertional portion can be configured to be inserted into the ear canal. The insertional portion can define a channel therethrough from the insertional end to the outer end. A flange portion can extend outwardly from the insertional portion at the outer end of the insertional portion.

### CROSS-REFERENCE TO RELATED APPLICATIONS

This application claims priority to U.S. Provisional Application No. 63/170,132, filed Apr. 2, 2021, and U.S. Provisional Application No. 63/227,599, filed Jul. 30, 2021, the entirety of each of which, including the appendices, is hereby incorporated by reference herein.

### FIELD

This application relates to ear canal stents and, in particular, to stents that are for inserting within an ear canal.

### BACKGROUND

Collapsing ear canals (e.g., bilateral collapsing ear canals) can lead to conductive hearing loss. The ear drum can function normally, yet the hearing is diminished. Surgery (e.g., canaloplasties) offer one remedy, but scarring and stenosis are common side effects to surgery, in addition to continuing issues with collapse. Thus, a device and method for supporting the ear canal to inhibit scar development and stenosis that occlude the ear canal is desirable.

### SUMMARY

Disclosed herein, in one aspect, is an ear stent that can be inserted into a collapsing ear canal to improve conductive hearing.

In one aspect, an ear stent can be configured for insertion into an ear canal of a user. The ear stent can have a longitudinal axis. The ear stent can comprise an insertional portion that extends along the longitudinal axis between an insertional end and an outer end. The insertional portion can be configured to be inserted into the ear canal. The insertional portion can define a channel therethrough from the insertional end to the outer end. A flange portion can extend outwardly from the insertional portion at the outer end of the insertional portion.

In one aspect, an ear stent for insertion into an ear canal of a user has a longitudinal axis. The ear stent includes an insertional portion that extends along the longitudinal axis between an insertional end and an outer end. The insertional portion is configured to be inserted into the ear canal. The insertional portion defines a channel therethrough from the insertional end to the outer end. The ear stent further includes a flange portion that extends outwardly from the insertional portion at the outer end of the insertional portion. The insertional portion and the flange portion are monolithically formed as a unitary body. The ear stent does not comprise any electronic components.

In one aspect, a kit comprises a plurality of ear stents. Each ear stent of the plurality of ear stents has an insertional length from the insertional end of the insertional portion to the flange portion, and an outer surface that has a cross-sectional shape in a cross sectional plane that is transverse to the longitudinal axis, wherein the cross-sectional shape has a major dimension. Each ear stent of the plurality of ear stents differs from each other ear stent of the plurality of ear stents in at least one of an insertional length, a cross sectional shape, or a major dimension.

In one aspect, a method comprises inserting an ear stent into an ear canal of a user, the ear stent having a longitudinal axis. The ear stent includes insertional portion that extends along the longitudinal axis between an insertional end and an outer end. The insertional portion is configured to be inserted into the ear canal. The insertional portion defines a channel therethrough from the insertional end to the outer end. The ear stent further includes a flange portion that extends outwardly from the insertional portion at the outer end of the insertional portion. The insertional portion and the flange portion are monolithically formed as a unitary body. The ear stent does not comprise any electronic components.

Additional advantages of the disclosed system and method will be set forth in part in the description which follows, and in part will be understood from the description, or may be learned by practice of the disclosed system and method. The advantages of the disclosed system and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed apparatus, system, and method and together with the description, serve to explain the principles of the disclosed apparatus, system, and method.

FIG. 1A shows a side view of an exemplary ear stent in accordance with embodiments disclosed herein. FIG. 1B shows a front view of the exemplary ear stent of FIG. 1A. FIG. 1C shows a perspective view of the exemplary ear stent of FIG. 1A. FIG. 1D shows an end view of the exemplary ear stent of FIG. 1A.

FIG. 2 shows a perspective view of a pair of ear stents as disclosed herein.

FIG. 3A shows an insertional end perspective view of an exemplary ear stent in accordance with embodiments disclosed herein. FIG. 3B shows a side elevational view of the ear stent of FIG. 3A. FIG. 3C shows an insertional end view of the ear stent of FIG. 3A. FIG. 3D shows an outer end of the ear stent of FIG. 3A.

FIG. 4A shows an insertional end perspective view of an exemplary ear stent in accordance with embodiments disclosed herein. FIG. 4B shows a side elevational view of the ear stent of FIG. 4A. FIG. 4C shows an insertional end view of the ear stent of FIG. 4A. FIG. 4D shows an outer end of the ear stent of FIG. 4A.

FIG. 5A shows an outer end perspective view of an exemplary ear stent in accordance with embodiments disclosed herein. FIG. 5B shows a side elevational view of the ear stent of FIG. 5A. FIG. 5C shows a front elevational view of the ear stent of FIG. 5A. FIG. 5D shows an insertional end view of the ear stent of FIG. 5A. FIG. 5E shows an outer end of the ear stent of FIG. 5A.

FIG. 6A shows an outer end perspective view of an exemplary ear stent in accordance with embodiments disclosed herein. FIG. 6B shows an insertional end perspective view of the ear stent of FIG. 6A. FIG. 6C shows a side elevational view of the ear stent of FIG. 6A. FIG. 6D shows a front elevational view of the ear stent of FIG. 6A. FIG. 6E shows an insertional end view of the ear stent of FIG. 6A. FIG. 6F shows an outer end of the ear stent of FIG. 6A.

FIG. 7A shows a perspective view of an exemplary ear stent in accordance with embodiments disclosed herein. FIG. 7B shows an outer end view of the ear stent of FIG. 7A. FIG. 7C shows a front elevational view of the ear stent of FIG. 7A. FIG. 7C shows a side elevational view of the ear stent of FIG. 7A.

FIG. 8A shows a perspective view of an exemplary ear stent in accordance with embodiments disclosed herein. FIG. 8B shows an outer end view of the ear stent of FIG. 8A. FIG. 8C shows a front elevational view of the ear stent of FIG. 8A. FIG. 8C shows a side elevational view of the ear stent of FIG. 8A.

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “a retention feature” includes one or more of such retention features, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Disclosed herein, in various aspects, is an ear stent. The ear stent can be inserted into an ear canal. The ear stent can be a non-surgical solution for a collapsed, or collapsing, ear canal. For example, the ear stent can maintain the ear canal in an open configuration. In this way, the ear stent can allow fluid communication through the ear canal. Thus, the ear stent can mitigate conductive hearing loss due to the collapsed ear canal, thereby improving the quality of life of the user.

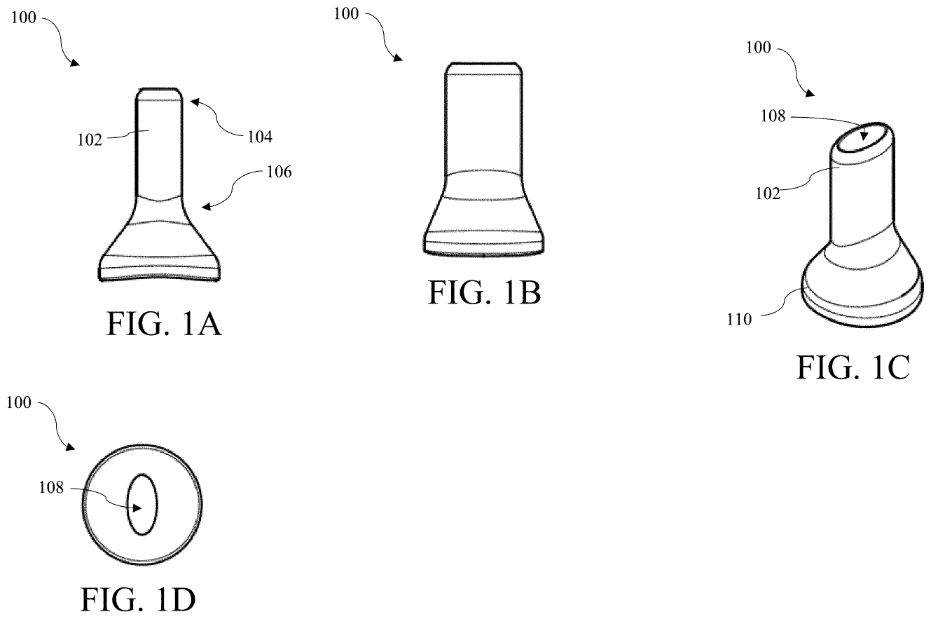

Referring to FIGS. 1-6F, according to various aspects, an ear stent 100 can have a longitudinal axis 101. The ear stent 100 can comprise an insertional portion 102 that extends along the longitudinal axis 101 between an insertional end 104 and an outer end 106. As used herein, the term “insertional” can optionally refer to portions of the ear stent 100 that are configured for at least partial insertion within a portion of the ear canal. The insertional portion 102 can define a channel 108 therethrough from the insertional end to the outer end. The channel 108 can communicate sound through the ear stent 100. It is contemplated that the channel 108 can have a maximum cross sectional area in planes transverse to the longitudinal axis 101 in view of limitations such as minimum wall thickness for structural integrity of the ear stent 100. Optionally, the channel 108 can have circular cross sections in planes transverse to the longitudinal axis 101. The circular cross sections of the channel 108 can optionally have a diameter ranging from about 1.2 mm to about 1.8 mm (e.g., about 1.5 mm). The diameter can be understood to be the major dimension of the circular cross section. In further aspects, the channel 108 can have an oval-shaped cross-sectional profile. In further aspects, the channel 108 can be half-circular or D-shaped (comprising a flat portion and an arcuate portion) in cross sectional planes transverse to the longitudinal axis 101.

The ear stent 100 can further comprise a flange portion 110 that extends radially outwardly from the insertional portion 102 at the outer end 106 of the insertional portion. The flange portion 110 can serve as a stop that prevents over-insertion into the ear canal. Thus, the flange can have a dimension transverse to the longitudinal axis 101 that inhibits insertion into the ear canal. Accordingly, in use, it is contemplated that the flange portion 110 (and any portions of the ear stent 100 positioned outwardly of the flange portion) will remain outside the ear canal. Optionally, the flange portion 110 can have a transverse dimension (e.g., diameter) ranging from about 8 mm to about 12 mm (e.g., about 10 mm). The insertional portion 102 can be configured to be at least partially (optionally, entirely) inserted into an ear canal (e.g., up to a point at which the flange portion 110 biases against the ear).

In various optional aspects, the insertional portion 102 and the flange portion 110 can be monolithically formed as a unitary body. In further aspects, the flange portion 110 and the insertional portion 102 can be formed separately and then coupled to each other.

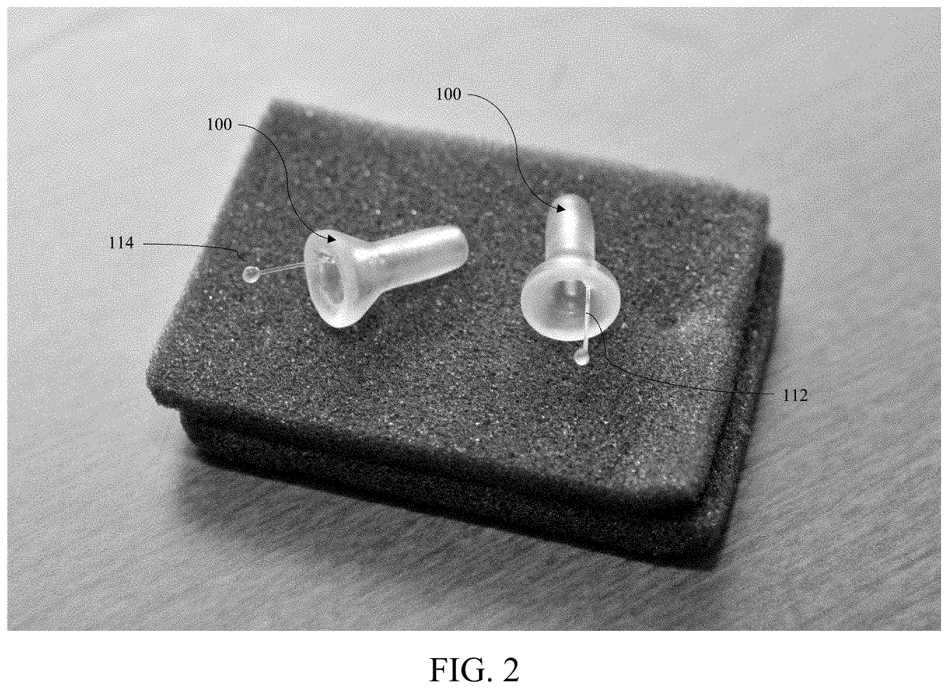

As shown in FIG. 2, the ear stent 100 can further comprise a protrusion 112 that extends outwardly from the flange along the longitudinal axis 101. The protrusion 112 can be used to pull the ear stent from the ear canal. Accordingly, the protrusion 112 can extend a sufficient distance for a user to grip and pull the ear stent from the ear canal. The sufficient distance can be, for example, at least 5 mm, at least 8 mm, or at least 10 mm. The protrusion 112 (FIG. 2) can optionally have an enlarged distal end 114 to facilitate gripping.

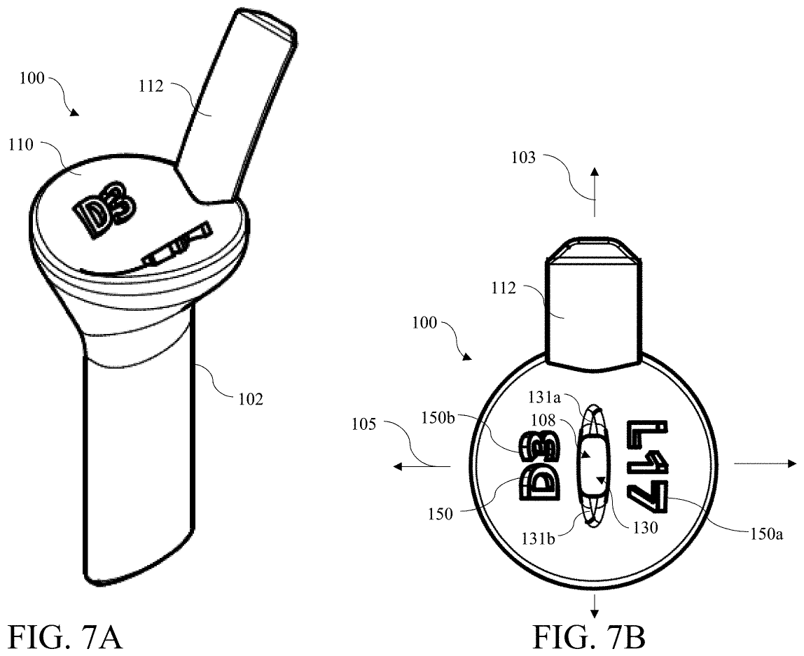

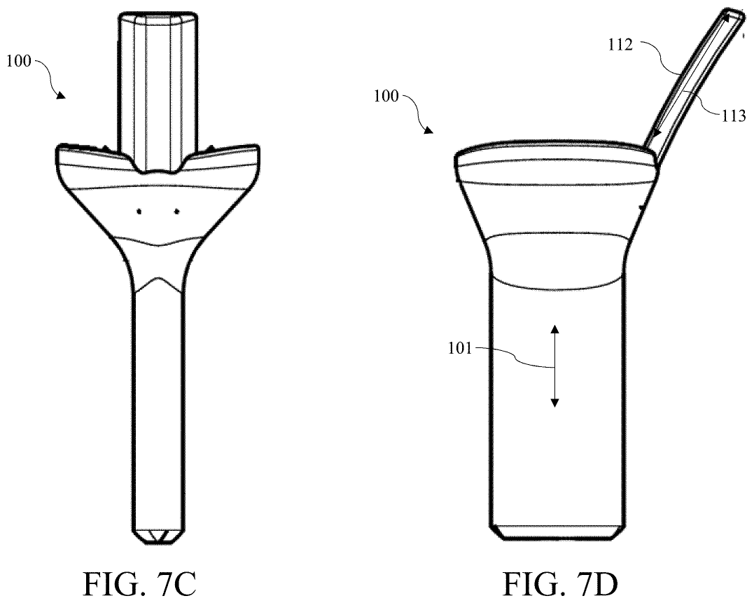

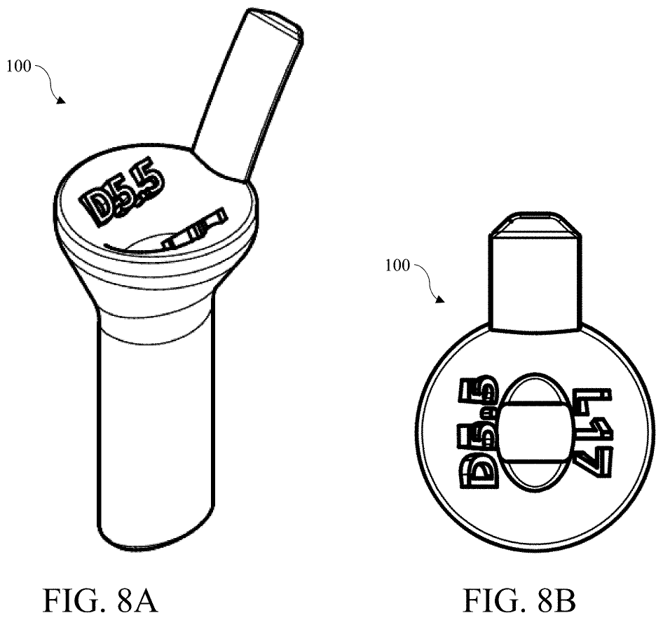

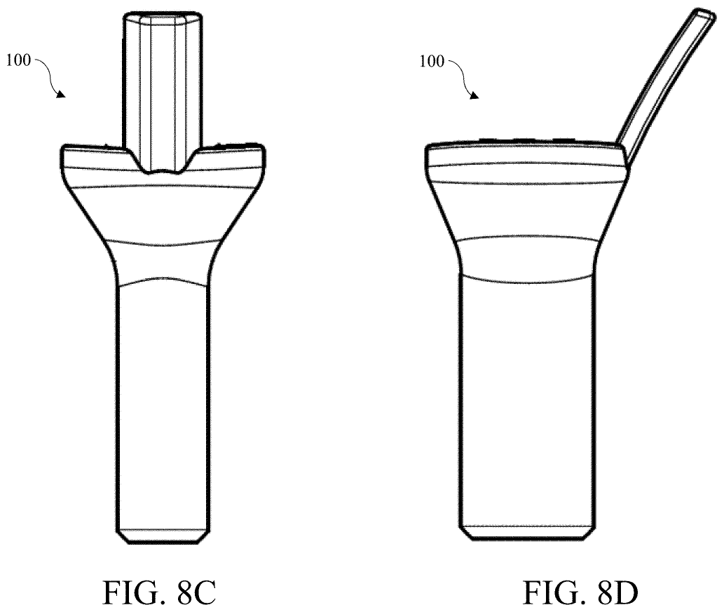

In further aspects, and as illustrated in FIGS. 7A-8D, the protrusion 112 can be integrally formed with the flange portion 110 and the insertional portion 102. For example, the protrusion 112 can be integrally formed with the flange portion 110 and the insertional portion 102 as a unitary, monolithic structure. Accordingly, in some optional aspects, the entire ear stent 100 can be formed by injection molding. The protrusion 112 can be elongate along an axis 113. In various aspects, the axis 113 can be straight or curved (e.g., arcuate). In some optional aspects, the axis 113 can extend at an acute angle relative to the longitudinal axis 101. Optionally, the protrusion 112 can have trapezoidal or substantially trapezoidal cross sections in planes perpendicular to the axis 113.

In some optional aspects, the protrusion 112 (FIG. 2) can be coupled to the insertional portion 102 and/or the flange portion 110 of the ear stent 100. For example, the ear stent 100 can define an opening 116 that extends longitudinally into the ear stent. In some optional aspects, the insertional portion 102 of the ear stent 100 can define the opening 116. In various optional aspects, the opening 116 can extend partially or entirely through the insertional portion 102 of the ear stent 100 along the longitudinal axis 101. For example, the channel 108 can comprise an open portion 130 and a filled portion 131, and the opening 116 can extend through the filled portion. In this way, the opening 116 does not reduce the wall thickness of the insertional portion 102 to compromise the structural integrity of the ear stent. A portion of the protrusion 112 can be received into the opening and coupled to the insertional portion 102. For example, an adhesive can couple the protrusion 112 to the insertional portion 102 in the opening 116. In some optional aspects, said adhesive can be the same material as the material of the insertional portion 102. For example, the insertional portion 102 (and, optionally, the flange portion 110) can be formed from acrylic material, and the acrylic material can fill the opening 116 around the protrusion 112 and then be allowed to cure. The opening 116 can optionally have a diameter of less than 1 mm (e.g., about 0.8 mm).

In further aspects, the protrusion 112 can be coupled to a surface of the insertional portion 102 or the flange 110. For example, in exemplary aspects, the protrusion 112 can be coupled (e.g., adhered) to an inner surface of the flange 110. In further aspects, the protrusion 112 can be coupled (e.g., adhered) to the channel 108 (e.g., along the inner surface of the insertional portion that defines the channel). In yet further aspects, the protrusion 112 can be integrally formed with the flange 110 and/or the insertional portion 102.

In optional aspects, the protrusion 112 can comprise monofilament. Optionally, the enlarged distal end 114 of the protrusion 112 can comprise polymer (e.g., acrylic) coupled to the monofilament.

In various aspects, the ear stent 100 can have an insertional length, Lᵢ, from the insertional end 104 of the insertional portion 102 to the flange portion 110. The insertional length L can optionally be from about 10 mm to about 20 mm, or from about 12 mm to about 18.5 mm, or about 10 mm or about 11 mm or about 12 mm or about 13 mm or about 14 mm or about 15 mm or about 16 mm or about 17 mm or about 18 mm or about 18.5 mm or about 19 mm or about 20 mm. Accordingly, in various optional aspects, the ear stent can have an overall length, L, be between 19 and 26 mm in length along the longitudinal axis 101.

Optionally, the length of the ear stent can be trimmed to fit to a particular ear canal of a user. It is contemplated that the ear stent can comprise a material (e.g., silicone) that allows the ear stent to be trimmed to a particular length while providing a comfortable fit.

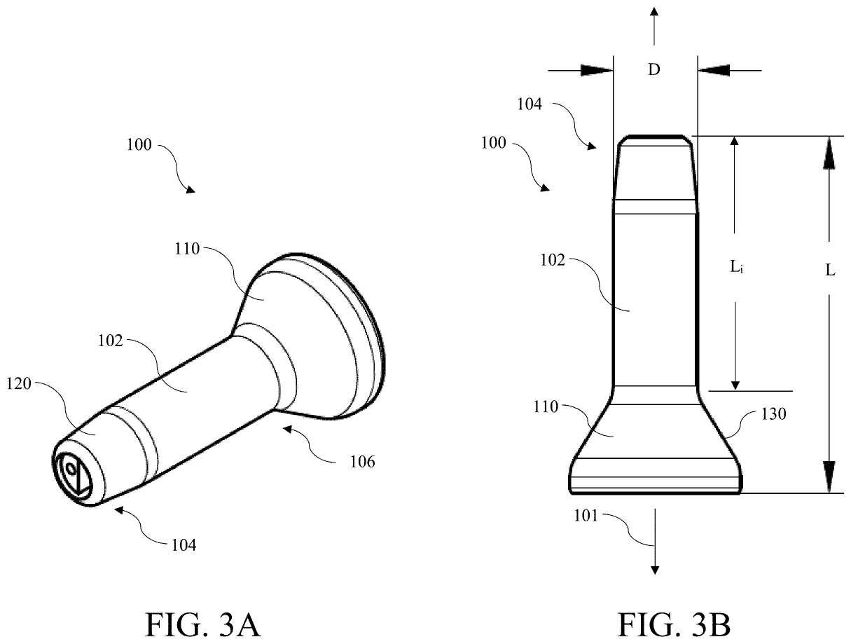

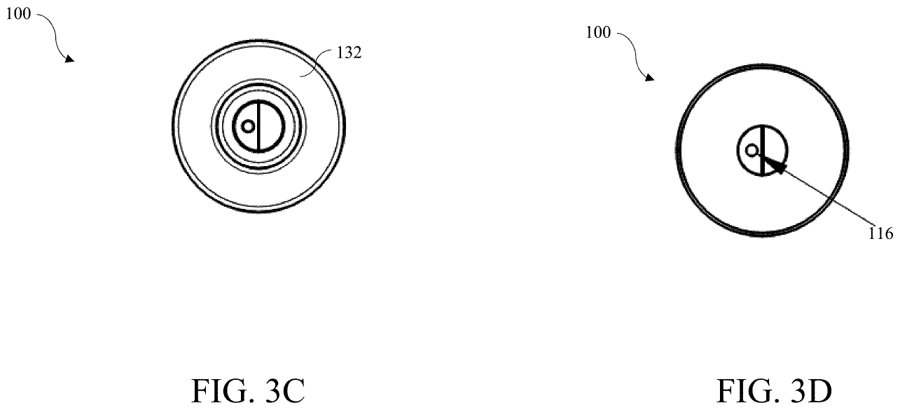

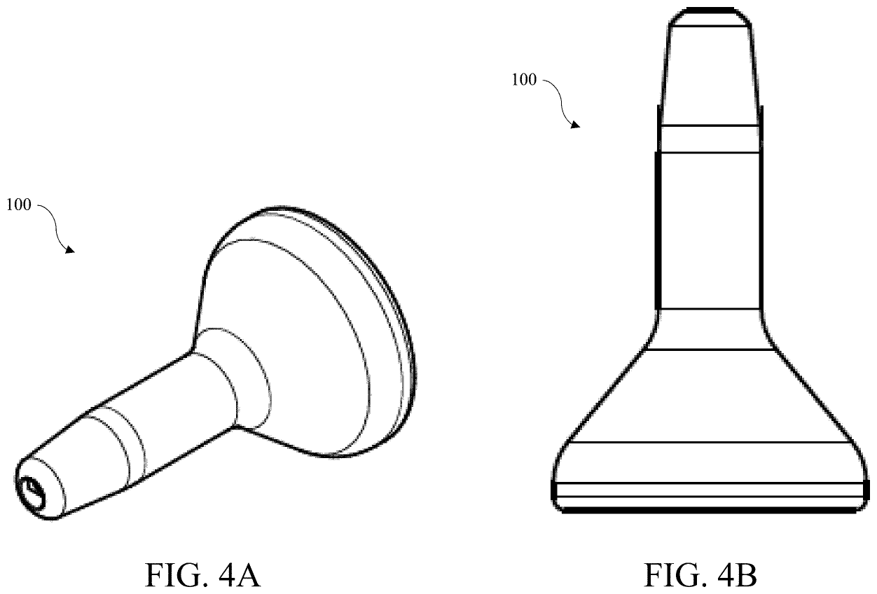

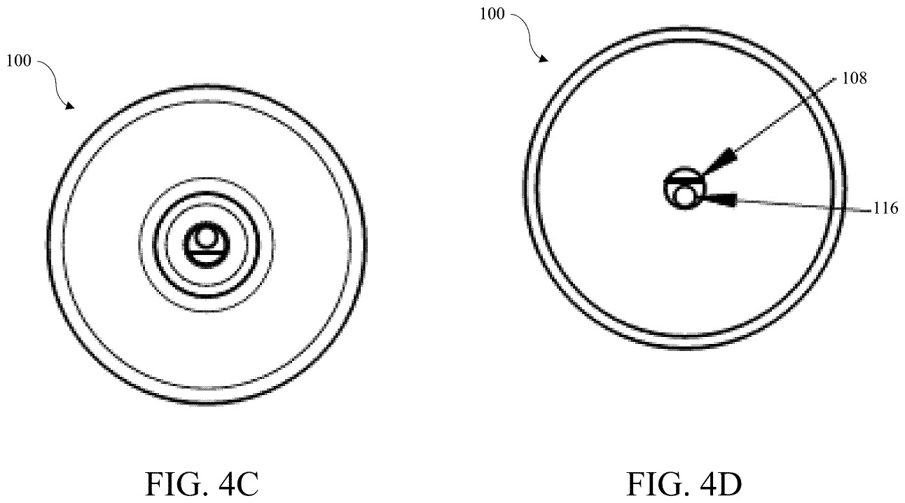

Referring to FIGS. 3A-4D, the insertional portion 102 of the ear canal can have an outer surface 118 that is configured to be received comfortably within the ear canal. For example, in some optional aspects, the outer surface 118 of the insertional portion 102 can be circular or generally circular in at least one cross sectional plane that is transverse to the longitudinal axis. In various optional aspects, the outer surface 118 can have a diameter, D, in the cross sectional plane that is from about 3 mm to about 7 mm, or about 4 mm to about 6 mm, or about 3 mm or about 4 mm or about 5 mm or about 6 mm or about 7 mm.

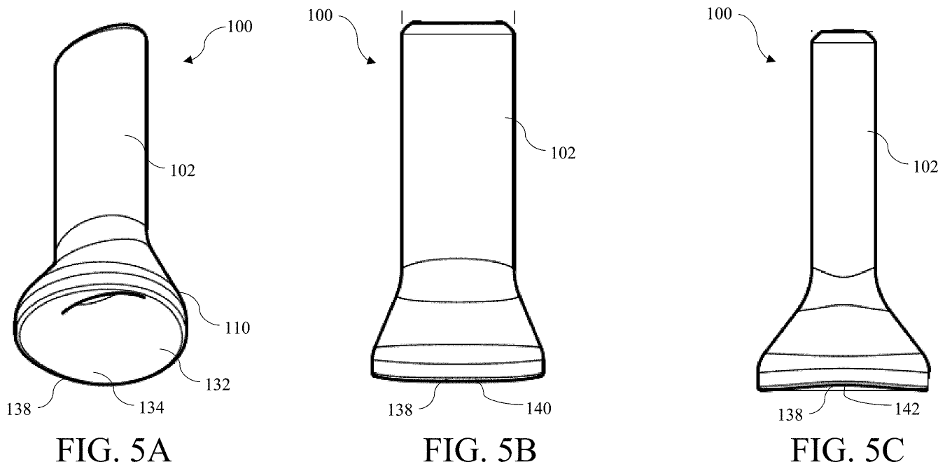

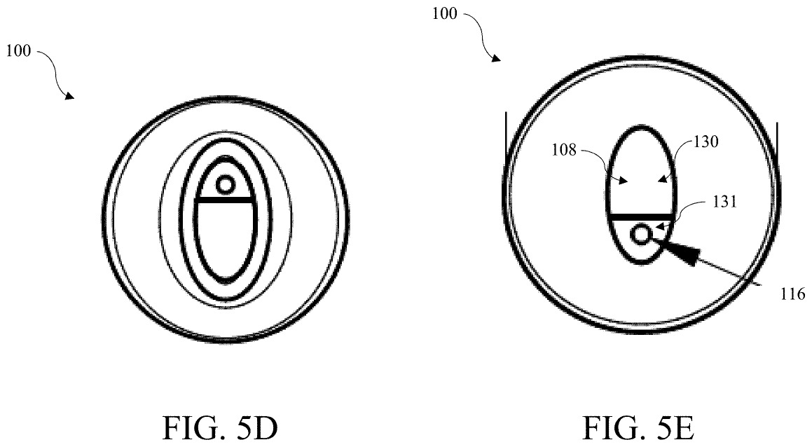

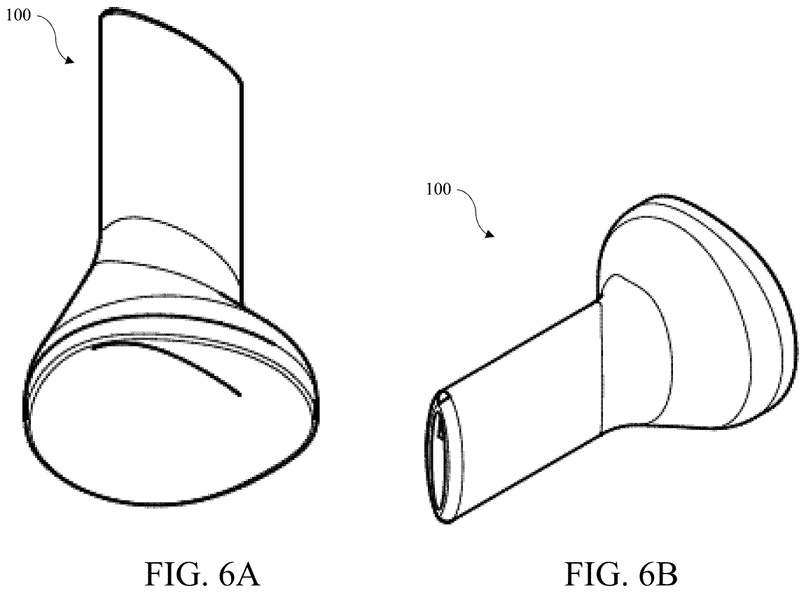

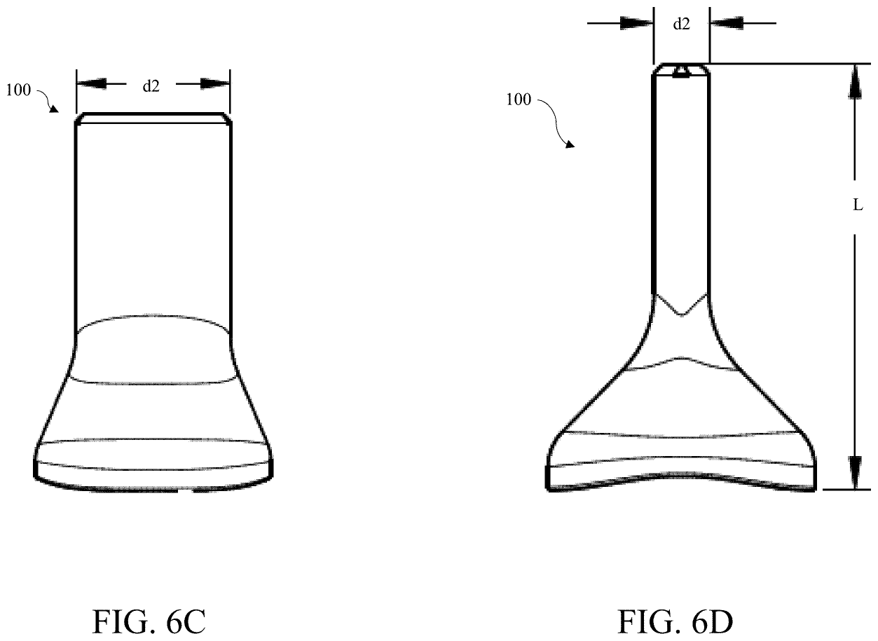

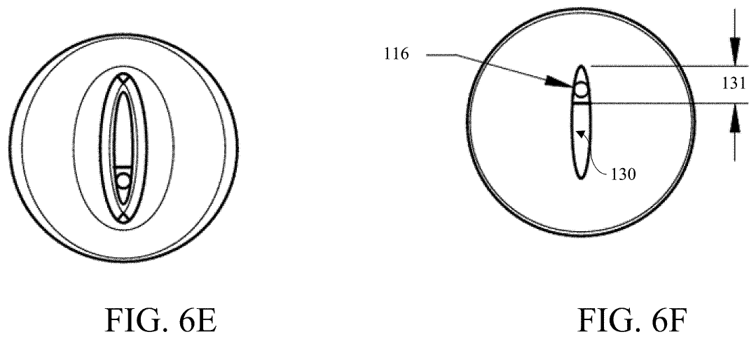

Referring to FIGS. 5A-6F, in further aspects, the outer surface 118 can have an oblong shape in at least one cross sectional plane that is transverse to the longitudinal axis 101. For example, the oblong shape can be an oval, an ellipse, an egg shape, or a more abstract shape. The oblong shape can have a major dimension, d1, and a minor dimension, d2. In various aspects, the major dimension can be from about 6 mm to about 9 mm or about 6 mm or about 6.5 mm or about 7 mm or about 7.5 mm or about 8 mm or about 8.5 mm or about 9 mm. In various aspects, the minor dimension, d2, can be from about 2 mm to about 6 mm or from about 2 mm to about 5 mm, or from about 2.5 mm to about 4.5 mm, or about 2 mm or about 2.5 mm or about 3 mm or about 3.5 mm or about 4 mm or about 4.5 mm or about 5 mm, or about 5.5 mm or about 6 mm.

In exemplary aspects, the ear stent 100 can have a wall thickness from about 1 mm to 2 mm (e.g., about 1.5 mm). Thus, the channel 108 can have a cross sectional profile (in planes perpendicular to the longitudinal axis 101) that corresponds to cross sections (in said plane) of the outer surface 118 inset by the wall thickness. Thus, for example, when the ear stent has a wall thickness of 1.5 mm, the outer surface 118 can have a major dimension of 8 mm and a minor dimension of 5.5 mm, and the channel 108 can have a major dimension of 5 mm and a minor dimension of 2.5 mm.

In further aspects, the insertional portion 102 of the ear stent 100 can have an increased wall thickness in certain regions to support the structure of the ear stent and inhibit restriction of the channel 108 due to compressive forces on the ear stent. In this way, the ear stent 100 can be formed from softer materials, such as silicone. As the outer surface 118 is selected for fitting the user's ear canal, the increased wall thickness can be provided by narrowing the channel. For example, in some aspects, and with reference to FIGS. 7B and 8B, the channel 108 can comprise an open portion 130 and opposed filled portions 131a,b (regions of increased wall thickness) that provide rigidity to the structure of the ear stent. In exemplary aspects, the channel 108 can be elongate along a first transverse axis 103 that is perpendicular to the longitudinal axis 101, and the filled portions 131a,b can be spaced along the transverse axis 103. The filed portions 131a,b can extend between opposed sides of the channel 108 along a second transverse axis 105 that is perpendicular to the transvers axis 103 and the longitudinal axis 101 to form the increased wall thickness of the insertional portion 102. As used herein, filled portions can be formed at the time of formation of the ear stent (e.g., during molding). Thus, the filled portions need not be material later added into the channel 108 after formation of the ear stent. Optionally, the filled portions 103a,b can be recessed from an inner surface 132 and/or outer surface of the flange.

It is contemplated that the profile of the insertional portion 102 can determine orientation of the ear stent 10 when in the ear. For example, the insertional portion 102 having oblong cross sections can be received within the ear with the transverse axis 103 in a generally vertical orientation. The protrusion 112 can be positioned so that, when inserted into the ear, the protrusion 112 is at the bottom of the ear stent. In this way, the protrusion 112 can guide the user when positioning the ear stent. Further, in this position, the protrusion 112 can generally be hidden, which can be desirable for aesthetics. Thus, for embodiments with the insertional portion 102 having an oblong cross sectional profile with a major dimension aligned with the first transverse axis 103, the protrusion 112 can be positioned toward or at an end of the flange 110 along the transverse axis. In some aspects, the first transverse axis 103 can intersect the projection 112. For example, as illustrated in FIG. 7, the protrusion 112 can be bisected by a plane that includes the first transverse axis 103 and the longitudinal axis 101. Accordingly, the outer surface 118 of the insertional portion 102 can define, in a cross sectional plane that is transverse to the longitudinal axis, an oblong shape having a major dimension and a minor dimension, with the major dimension extending along the transverse axis 103 that is perpendicular to the longitudinal axis, and the protrusion 112 can be offset from the longitudinal axis 101 along the transverse axis.

Optionally, the outer surface 118 of the insertional portion 102 can have consistent cross sections along at least a portion of its length along the longitudinal axis 101 (e.g., at least half of its length, at least 60% of its length, or at least 75% of its length). In some optional aspects, the outer surface 118 of the insertional portion 102 can define a taper 120 toward the insertional end 104. The taper 120 can optionally start between the insertional end 104 and the outer end 106 (e.g., at less than 5 mm or at less than 10 mm from the insertional end). In further aspects, the insertional portion 102 can define a continuous taper from the outer end to the insertional end. In some optional aspects, the insertional end 104 of the insertional portion 102 can have rounded surfaces 122. Thus, in various optional aspects, the insertional portion 102 can be free of sharp edges.

In some optional aspects, the flange 110 can have an outer surface 130 that tapers toward the insertional end 104 of the insertional portion 102. In various optional aspects, the flange 110 can have an inner surface 132 that defines a funnel 134 that tapers along the longitudinal axis toward the channel 108. Optionally, the flange 110 can define an outer rim 138 that defines opposing convex portions 140 and opposing concave portions 142.

The ear stent 100 can optionally comprise at least one retention feature. One exemplary retention feature can be, for example, a hook that extends around the ear of the user. Optionally, said hook can have a similar profile to earhooks that are conventionally used for hearing aids. Another exemplary retention feature can be a hook that is tucked in to the ear (e.g., against the antihelix). In further aspects, ear stent 100 can omit any retention feature, or, optionally, omit any retention feature that extends around the ear. It is contemplated that omission of the retention feature, and, in particular, a retention feature that extends around the ear, can make the ear stent more aesthetically pleasing.

The ear stent 100 can optionally comprise polymer, such as, for example, clear biocompatible resin (e.g., FORMLABS BIOMED clear biocompatible resin). Optionally, the ear stent can comprise a latticed material. In this way, the ear stent can provide for aeration of the ear canal. Further, the latticed material can wick antibiotics or other fluids (e.g., drops) to the outer surfaces of the ear stent to permit drug delivery within the ear, thereby permitting treatment of fungal infections and other conditions within the ear without requiring removal of the ear stent. In further aspects, the ear stent can comprise a material that expands upon exposure to moisture to bias outwardly against the ear. In yet further optional aspects, the ear stent can comprise a compressible material that is configured to at least partially conform to a shape of the ear canal while having sufficient structural rigidity to maintain the channel 108 in an open configuration through the ear canal. In exemplary aspects, the material can have Shore A hardness of greater than 50 (e.g., optionally, between 60-100, or between 75 and 95, or from about 80 to about 90). In further aspects, the material can have a Shore D hardness from about 0 to about 90, or from 0 to about 80, or about 75 to about 85 (e.g., about 78). In some optional aspects, the ear stent 100 can be formed from a soft material such as silicone. For example, the ear stent 100 can consist of, or consist essentially of, silicone.

At least a portion of (or an entirety of, or substantially an entirety of) an outer surface of the ear stent can be smooth. For example, the outer surface can be sanded with 1000 grit sandpaper.

In some optional aspects, it is contemplated that the ear stent 100 can be free of electronics. In this way, the ear stent 100 can be inexpensively manufactured. For example, as further described herein, in some aspects, each ear stent can be manufactured via 3D printing for under a dollar (e.g., about $0.64).

Although embodiments herein show the ear stent as having a straight longitudinal axis, it is contemplated that further embodiments can have a curved longitudinal axis.

The ear stent 100 can be held in place by the collapsing canal (e.g., the cartilage of the distal external auditory canal).

In various aspects, the ear stent is not surgically implanted. Instead, the ear stent can be manually inserted into the ear canal (e.g., optionally, by the user). The ear stent can optionally be removed. For example, the user or a clinician can grip the protrusion 112 to pull the ear stent 100 from the ear canal.

In some optional aspects, the ear stent 100 can be formed via molding (e.g., injection molding). For example, the ear stent can be formed from molded silicone. In further optional aspects, the ear stent 100 can be formed via additive manufacturing (e.g., using 3D printing). It is contemplated that ear stents comprising harder durometer material than silicone can be formed using additive manufacturing. The ear stent can be configured for receipt within the ear of a particular user. For example, a mold of the ear canal can be taken (e.g., via silicone impression), and the ear stent can be formed based on at least a portion of a profile of the mold. In various aspects, the mold can be formed by 3D printing or by machining.

In further aspects, the ear stent can be selected from a kit comprising a plurality of ear stents so that the ear stent comfortably and effectively fits the particular user. For example, each ear stent of the plurality of ear stents can vary from the other ear stents of the plurality of ear stents in at least one of insertional length, the cross sectional shape, cross-sectional area, material, material properties (e.g., wicking properties and/or durometer), wall structure (e.g., solid or lattice), or transverse dimension (e.g., major dimension (optionally, diameter) or minor dimension of the shape of the outer surface of the insertional portion of the ear stent in a plane transverse to the longitudinal axis). For example, the kit can comprise ear stents with two different cross sectional shapes (e.g., oval and circular), different transverse dimensions (e.g., three different diameters or major or minor dimensions), and a plurality of different lengths for each cross sectional shape and size (e.g., between 2 and 10 different lengths, or about 7 different lengths (for example, a series of lengths that are provided in increments of 1 mm). In one example, the kit can comprise a plurality of stents having circular cross-sections and a plurality of stents having oval cross-sections, with the stents having circular cross-sections being provided in a plurality of diameters and the stents having oval cross-sections being provided with a consistent major dimension and different minor dimensions. More generally, it is contemplated that the kit can comprise ear stents that provide any desired combination of lengths, cross-sectional shapes, cross-sectional areas, materials, material properties, wall structures, or transverse dimensions. In exemplary aspects, the kit can comprise a carrier (e.g., a box or other receptacle) for receiving and transporting the plurality of ear stents. In some aspects, the carrier can comprise different receptacles (e.g., separated by dividers) that allow for organizing ear stents based on different properties, such as length, cross section, material, etc.

In one aspect, a kit can comprise a plurality of ear stents that vary in different combinations of length and different combinations of major dimensions. For example, in some aspects, the kit can comprise a plurality of ear stents 100 having different lengths (e.g., six different insertional lengths), and for each length, the kit can comprise a plurality of different cross sectional profiles. Optionally, the different cross sectional profiles can all be oblong (e.g., oval), having consistent major dimensions and differing minor dimensions. In exemplary aspects, the kit can comprise 36 different varieties of ear stent, including six different cross sectional profiles, with each different cross sectional profile being provided in six different insertional lengths. For example, the kit can comprise ear stents having oval cross sections with a major dimension of 8 mm and minor dimensions of 3 mm, 3.5 mm, 4 mm, 4.5 mm, 5 mm, and 5.5 mm. In each of said cross sections, individual ear stents can have insertional lengths of 12 mm, 13 mm, 14 mm, 15 mm, 16 mm, and 17 mm, respectively. In various aspects, the kit can comprise ear stents having from 4 to 10 different insertional lengths, with the stents of a given insertional length having from 4 to 10 different cross sectional profiles.

In some optional aspects, the ear stents 100 of the kit can all have the same wall thickness. In further optional aspects, the ear stents 100 of the kit can have projections 112 that are all the same size for each stent.

The ear stents can comprise indications 150 of their dimensions. For example, the indications 150 can be formed into the ear stent (e.g., into a portion of the flange configured not to engage the ear). As illustrated, the indications 150 can comprise a first indication 150a showing an insertional length of the ear stent (shown, for example, in millimeters) and a first indication 150b showing a cross sectional dimension (e.g., a minor dimension of the cross section, shown in millimeters). In some aspects, the indications 150 can be incorporated into a mold that forms the ear stent so that the indications are provided upon formation of the ear stent. In further aspects, for ear stents that are 3D printed, the indications can be 3D printed as part of the ear stent. The indications can optionally be on the outwardly facing surface of the flange. In this way, the indications can be easily visible and do not change the profile of the surfaces of the ear stent that contact the ear canal.

In exemplary aspects, an ear stent having a particular length and cross-section can be provided in multiple forms having different softness and hardness (e.g., varying durometer). For example, the ear stent can be provided as a hard (generally rigid) body and a soft body. It is contemplated that a hard material can be selected for temporary use, such as for post-surgical applications, whereas a softer material can be selected for more permanent applications, such as a collapsing ear canal. Accordingly, in some aspects, the kit can comprise ear stents of the same size and shape that are formed from different materials (e.g., having different durometer measurements). In further aspects, different kits can have different material properties. For example, a post-surgical kit can comprise hard material, whereas a collapsing ear canal kit can comprise soft material. In some aspects, a kit can comprise both hard and soft materials. For example, a kit can comprise different varieties of ear stent, including a plurality of different cross sectional profiles, with each different cross sectional profile being provided in a plurality of different insertional lengths, and with each combination of cross sectional profile and length provided in both a hard material and a soft material. Thus, for example, a kit can comprise 72 different varieties of ear stent, including six different cross sectional profiles, with each different cross sectional profile being provided in six different insertional lengths, and with each combination of cross sectional profile and length provided in both a hard material and a soft material.

In fitting a user with an ear stent, it is contemplated that the ear stent can be selected so that it extends about halfway, or at least halfway through the ear canal (i.e., between the tympanic membrane and the opening of the outer ear). For example, the insertional end of the insertional portion of the ear stent can be short of the bony portion of the ear canal to provide optimal comfort. When a kit as disclosed herein is employed, it is contemplated that multiple ear stents can be fitted within the ear canal of the subject until the ear stent providing the optimal fit is identified. In exemplary aspects, it is contemplated that the ear stents disclosed herein can allow a clinician to look through the central opening of the stents to visualize the location of the insertional end of the stent and evaluate the quality of the fit within the ear canal. Optionally, it is contemplated that the ear stents can comprise transparent or at least partially transparent materials to provide improved visualization.

Although the ear stents are described herein for use in maintaining open the external ear canal, the ear stents can be used in various other applications and for individuals having various conditions that can be improved from such structural support. For example, the ear stent can be used in post-surgical patients to maintain the ear canal open to allow the soft tissue to heal. In post-surgical situations, for example, the soft tissue can be maintained in compression so that the soft tissue does not extend into and constrict the ear canal. In further aspects, the ear canal can be used to reshape or adjust the geometry of the ear canal. For example, the ear canal can be stretched or reshaped before surgery. In yet further aspects, the ear stent can maintain the ear canal open during acute swelling due to instances such as inflammation, infection, or trauma. In various aspects, the disclosed ear stent can be used for chronic inflation disease, acquired stenosis, or after surgery, to hold the ear canal in place after surgical procedures.

Except where otherwise indicated, although the disclosed ear stents are depicted in the drawings as having particular shapes, it is understood that the functional features disclosed herein can be achieved with embodiments having different shapes than those depicted in the drawings. It is contemplated that the ear stents depicted in the drawings can achieve the functional advantages disclosed herein while also serving an ornamental purpose.

Although specific geometries are described, it is contemplated that other geometries can be used to achieve the disclosed functions of the ear stents, and it is further contemplated that the disclosed geometries can serve both a functional and an ornamental purpose.

Those skilled in the art will recognize, or be able to ascertain using no more than routine experimentation, many equivalents to the specific embodiments of the method and compositions described herein. Such equivalents are intended to be encompassed by the following claims.

What is claimed is:

1. An ear stent for insertion into an ear canal of a user, the ear stent having a longitudinal axis and comprising:
an insertional portion that extends along the longitudinal axis between an insertional end and an outer end, wherein the insertional portion is configured to be inserted into the ear canal, wherein the insertional portion defines a channel therethrough from the insertional end to the outer end; and
a flange portion that extends outwardly from the insertional portion at the outer end of the insertional portion, wherein the insertional portion and the flange portion are monolithically formed as a unitary body,
wherein the ear stent does not comprise any electronic components.

2. The ear stent of claim 1, further comprising a protrusion that extends outwardly from the flange along the longitudinal axis a sufficient distance for a user to grip to pull the ear stent from the ear canal.

3. The ear stent of claim 2, wherein the sufficient distance is at least 5 mm.

4. The ear stent of claim 2, wherein the protrusion is integrally formed with the flange portion as a unitary, monolithic structure.

5. The ear stent of claim 2, wherein the insertional portion has an outer surface that defines, in a cross sectional plane that is transverse to the longitudinal axis, an oblong shape having a major dimension and a minor dimension, wherein the major dimension extends along a transverse axis that is perpendicular to the longitudinal axis, wherein the protrusion is offset from the longitudinal axis along the transverse axis.

6. The ear stent of claim 5, wherein the protrusion extends along an axis that forms an oblique angle with the longitudinal axis.

7. The ear stent of claim 1, wherein the ear stent has an insertional length from the insertional end of the insertional portion to the flange portion, wherein the insertional length is between 10 mm and 20 mm.

8. The ear stent of claim 1, wherein the insertional portion has an outer surface that defines, in a cross sectional plane that is transverse to the longitudinal axis, an oblong shape having a major dimension and a minor dimension.

9. The ear stent of claim 8, wherein the major dimension is between 6 mm and 9 mm.

10. The ear stent of claim 8, wherein the minor dimension is between 2 mm and 5 mm.

11. The ear stent of claim 1, wherein the insertional portion defines a taper toward the insertional end.

12. The ear stent of claim 1, wherein the flange portion defines a taper toward the insertional end of the insertional portion.

13. The ear stent of claim 1, wherein the flange portion has an inner surface that defines a funnel that tapers along the longitudinal axis toward the channel.

14. The ear stent of claim 1, were in the ear stent consists essentially of silicone.

15. A method comprising:
inserting an ear stent into an ear canal of a user, the ear stent having a longitudinal axis, wherein the ear stent comprises:
an insertional portion that extends along the longitudinal axis between an insertional end and an outer end, wherein the insertional portion is inserted into the ear canal, wherein the insertional portion defines a channel therethrough from the insertional end to the outer end; and
a flange portion that extends outwardly from the insertional portion at the outer end of the insertional portion, wherein the insertional portion and the flange portion are monolithically formed as a unitary body,
wherein the ear stent does not comprise any electronic components.

16. The method of claim 15, wherein the user has a collapsed or collapsing ear canal, and wherein the ear stent supports the ear canal of the user in an at least partially open condition.

17. A kit comprising:
a plurality of ear stents, wherein each ear stent of the plurality of ear stents has a longitudinal axis, wherein the each ear stent of the plurality of ear stents comprises:
an insertional portion that extends along the longitudinal axis between an insertional end and an outer end, wherein the insertional portion is configured to be inserted into the ear canal, wherein the insertional portion defines a channel therethrough from the insertional end to the outer end; and
a flange portion that extends outwardly from the insertional portion at the outer end of the insertional portion,
wherein each ear stent of the plurality of ear stents has:
an insertional length from the insertional end of the insertional portion to the flange portion, and
an outer surface that has a cross sectional shape in a cross sectional plane that is transverse to the longitudinal axis, wherein the cross sectional shape has a major dimension,
wherein each ear stent of the plurality of ear stents differs from each other ear stent of the plurality of ear stents in at least one of an insertional length, a cross sectional shape, or a major dimension.

18. The kit of claim 17, wherein the outer surface of the insertional portion of each ear stent of the plurality of ear stents defines, in a cross sectional plane that is transverse to the longitudinal axis, an oblong shape having a major dimension and a minor dimension, wherein the minor dimensions of the oblong shapes of the plurality of ear stents vary from about 3 mm to about 5.5 mm.

19. The kit of claim 17, wherein the insertional lengths of the plurality of ear stents vary from about 12 mm to about 17 mm.

20. The kit of claim 18, wherein the plurality of ear stents include ear stents having at least six different insertional lengths and at least six different minor dimensions.
